(19) United States
(12) Patent Application Publication
(10) Pub. No.: US 2005/0113723 A1
(43) Pub. Date: May 26, 2005

(54) Relaxation system, relaxation method, relaxation program, massage system, massage method, massage program, physical activity determiner, physical activity determination method, and physical activity determination program

(76) Inventors: Kenji Ueyama, Osaka (JP); Keiko Watanabe, Osaka (JP)

Correspondence Address: McDERMOTT WILL & EMERY LLP, 600 13th Street, N.W., Washington, DC, 20005-3096, US

(21) Appl. No.: 10/963,756

(22) Filed: Oct. 14, 2004

(30) Foreign Application Priority Data
Oct. 16, 2003 (JP) 2003-356568

(51) Int. Cl. A61M 21/00; A61H 15/00

(52) U.S. Cl. 601/15; 601/99; 601/103; 600/28

(57) ABSTRACT

A calorie meter which is attachable to a human body measures the acceleration of its attached part, and a CPU estimates activity information of the human body based on the measured acceleration. Based on the result of estimation, a massage operation is controlled by the control circuit of a massager.

### BACKGROUND OF THE INVENTION

1. Field of the Invention

The present invention relates to a relaxation system, relaxation method, relaxation program, massage system, massage method, massage program, physical activity determiner, physical activity determination method, and physical activity determination program.

2. Description of the Background Art

Various types of apparatus for providing massage to a human body have conventionally been developed. One such example is a massage chair that is provided with massaging members at the backrest of the chair seated by a user. This massage chair is capable of pressing the rear side of the user by allowing the vibrating massaging members to move upward, downward, leftward or rightward along the rear side of the seated user, such as neck, shoulders, back, and waist.

JP 2002-165856 A proposes a massage machine for automatically massaging various parts of the human body.

The massage machine as described in JP 2002-165856 A comprises a vital information sensor which detects the vital information on the autonomic nervous system of a person to be massaged, and a control circuit which controls the massage operation based on the vital information detected by the vital information sensor.

This control circuit estimates the psychological state of the person to be massaged based on variations in the vital information detected by the vital information sensor, to adjust the massage operation according to the estimated psychological state. For the adjustment of the massage operation, it is possible to select either a relaxed mode or refresh mode. In the relaxed mode, the massage operation is adjusted so as to decrease the activity of the autonomic nervous system, whereas in the refresh mode, the massage operation is adjusted so as to increase the activity of the autonomic nervous system.

Thus, the massage machine of JP 2002-165856 A can provide an effective massage to the person to be massaged according to the purpose of massage.

The above-described massage machine, however, has presented difficulty in providing even more effective massage, because the fatigue parts and degree of fatigue of a human body are varied from time to time, depending on its active parts and amount of activity. What is also demanded is to realize recovery from fatigue even more effectively in a shorter period of time.

### SUMMARY OF THE INVENTION

It is an object of the present invention to provide a relaxation system, relaxation method, and relaxation program which enable recovery from fatigue more effectively in a short period of time.

It is another object of the present invention to provide a massage system, massage method, and massage program which enable recovery from fatigue more effectively in a short period of time.

It is still another object of the present invention to provide a physical activity determination system, physical activity determination method, and physical activity determination program which enable determination of activity information.

A relaxation system according to one aspect of the present invention comprises: an acceleration measurement device that is attachable to a human body, and measures the acceleration of an attached part; a relaxation apparatus that performs a relaxation operation; an estimator that estimates activity information of the human body based on the acceleration measured by the acceleration measurement device; and a controller that controls the relaxation apparatus based on a result of estimation by the estimator.

In the relaxation system, the acceleration measurement device attachable to the human body measures the acceleration of its attached part, and the estimator estimates the active information of the human body based on the measured acceleration. Based on the result of estimation, the relaxation operation is controlled by the controller.

In this case, since the relaxation operation is controlled based on the estimation result of the activity information of the human body, recovery from fatigue is realized more effectively in a short period of time, according to the fatigue parts, working contents or degree of fatigue.

The activity information may include an active part of the human body. In this case, the active part is estimated, and the relaxation operation is controlled based on the result of estimation. This helps recovery from fatigue according to the fatigue parts more effectively in a short period of time.

The estimator may calculate a momentum variation based on the acceleration measured by the acceleration measurement device, to estimate the active information of the human body based on the calculated momentum variation.

In this case, since a momentum variation is correlated with active information of the human body, it is possible to easily estimate the active information of the human body based on the momentum variation by measuring in advance the correlation there between.

The relaxation apparatus may include a massage apparatus with a pressing member movably provided in order to press parts of the human body, and the controller may control at least either of the speed or time of the pressing member based on the result of estimation by the estimator.

In this case, since at least either of the speed or time of the pressing member is controlled based on the result of estimation of the activity information of the human body, the relaxation operation can be performed more effectively in a short period of time, according to the fatigue parts, working contents or degree of fatigue.

The relaxation apparatus may relieve stress visually, audibly or physically.

In this case, since the relaxation apparatus that relieves stress visually, audibly or physically is controlled based on the result of estimation of the activity information of the human body, not only the physical fatigue but also the mental fatigue may be recovered with the stress being relieved visually, audibly, or physically.

The relaxation apparatus may include one or more of an air conditioner, an audio apparatus, a video display, and an illuminator, and the controller may control the one or more of air conditioner, audio apparatus, video display, and illuminator based on the result of estimation by the estimator.

In this case, based on the result of estimation of the active information of the human body, one or more of the air conditioner, audio apparatus, video display, and illuminator are controlled. Thus, the video display and illuminator act to relieve stress visually, the audio apparatus acts to relieve stress audibly, or the air conditioner acts to relieve stress physically by controlling the temperature, humidity or air velocity. As a result, not only the physical fatigue but also the mental fatigue may be recovered.

The relaxation system may further comprise a detector that detects vital information of the human body, the controller setting the operation of the relaxation apparatus based on the vital information detected by the detector, to adjust the set operation based on the result of estimation by the estimator.

In this case, the operation of the relaxation apparatus is set based on the vital information, followed by adjustment of the set operation based on the result of estimation of the activity information of the human body; so that it is possible to help recovery from fatigue more effectively in a shorter period of time, considering the current body condition and fatigue parts of the human body, working contents or degree of fatigue.

The detector may include at least one of a galvanic skin response sensor, a pulse sensor, and a skin temperature sensor. In this case, at least one of the galvanic skin response sensor, pulse sensor, and skin temperature sensor enables detection of the current degree of stress for the human body.

The relaxation system may further comprise an attachment tool for use in attaching the acceleration measurement device around the waist of the human body.

In this case, acceleration is measured of the waist of the human body, followed by estimation of the active information based on the acceleration of the waist.

A relaxation method according to another aspect of the present invention comprises the steps of: measuring the acceleration of a human body; performing a relaxation operation; estimating active information of the human body based on the measured acceleration; and controlling the relaxation operation based on the result of estimation.

In the relaxation method, the acceleration of the human body is measured, followed by estimation of the active information of the human body based on the measured acceleration. The relaxation operation is controlled based on the result of estimation.

In this case, the relaxation operation is controlled based on the result of estimation of the activity information of the human body; so that it is possible to help recovery from fatigue more effectively in a short period of time according to the fatigue parts, working contents or degree of fatigue.

A relaxation program according to still another aspect of the present invention is a computer-executable relaxation program, which makes the computer to execute the processes of: measuring the acceleration of a human body; performing a relaxation operation; estimating activity information of the human body based on the measured acceleration; and controlling the relaxation operation based on the result of estimation.

In the relaxation program, the acceleration of the human body is measured, followed by estimation of the activity information of the human body based on the measured acceleration. Based on the result of estimation, the relaxation operation is controlled.

In this case, the relaxation operation is controlled based on the result of estimation of the activity information of the human body; so that it is possible to help recovery from fatigue more effectively in a short period of time according to the fatigue parts, working contents or degree of fatigue.

A massage system according to still another aspect of the present invention comprises: an acceleration measurement device that is attachable to a human body, and measures the acceleration of an attached part; a massage apparatus that performs a massage operation; an estimator that estimates activity information of the human body based on the acceleration measured by the acceleration measurement device; and a controller that controls the massage apparatus based on a result of estimation by the estimator.

In the massage system, the acceleration measurement device attachable to the human body measures the acceleration of its attached part, and the estimator estimates the active information of the human body based on the measured acceleration. Based on the result of estimation, the massage operation is controlled by the controller.

In this case, since the massage operation is controlled based on the estimation result of the activity information of the human body, recovery from fatigue is realized more effectively in a short period of time, according to the fatigue parts, working contents or degree of fatigue.

The massage apparatus may include a pressing member movably provided in order to press parts of the human body, and the controller may control at least either of the speed or time of the pressing member based on the result of estimation by the estimator.

In this case, since at least either of the speed or time of the pressing member is controlled based on the result of estimation of the activity information of the human body, a more effective massage can be performed in a short period of time, according to the fatigue parts, working contents or degree of fatigue.

The massage system may further comprise a relaxation apparatus that relieves stress visually, audibly, and physically, the controller controlling the relaxation apparatus based on the result of estimation by the estimator.

In this case, since the relaxation operation is controlled based on the estimation result of the activity information of the human body, recovery from fatigue is realized more effectively in a short period of time, according to the fatigue parts, working contents or degree of fatigue.

The relaxation apparatus may include one or more of an air conditioner, an audio apparatus, a video display, and an illuminator, and the controller may control the one or more of air conditioner, audio apparatus, video display, and illuminator based on the result of estimation by the estimator.

In this case, based on the result of estimation of the active information of the human body, one or more of the air conditioner, audio apparatus, video display, and illuminator are controlled. Thus, the video display and illuminator act to relieve stress visually, the audio apparatus acts to relieve stress audibly, or the air conditioner acts to relieve stress physically by controlling the temperature, humidity or air velocity. As a result, not only the physical fatigue but also the mental fatigue may be recovered.

The massage system may further comprise a detector that detects vital information of the human body, the controller setting the operation of the massage apparatus based on the vital information detected by the detector, to adjust the set operation based on the result of estimation by the estimator.

In this case, the operation of the massage apparatus is set based on the vital information, followed by adjustment of the set operation based on the result of estimation of the activity information of the human body; so that it is possible to help recovery from fatigue more effectively in a shorter period of time, considering the current body condition and fatigue parts of the human body, working contents or degree of fatigue.

The detector may include at least one of a galvanic skin response sensor, a pulse sensor, and a skin temperature sensor. In this case, at least one of the galvanic skin response sensor, pulse sensor, and skin temperature sensor enables detection of the current degree of stress for the human body.

A massage method according to still another aspect of the present invention comprises the steps of: measuring the acceleration of a human body; performing a massage operation; estimating active information of the human body based on the measured acceleration; and controlling the massage operation based on the result of estimation.

In the massage method, the acceleration of the human body is measured, followed by estimation of the active information of the human body based on the measured acceleration. The massage operation is controlled based on the result of estimation.

In this case, the massage operation is controlled based on the result of estimation of the activity information of the human body; so that it is possible to help recovery from fatigue more effectively in a short period of time according to the fatigue parts, working contents or degree of fatigue.

A massage program according to still another aspect of the present invention is a computer-executable massage program, which makes the computer to execute the processes of: measuring the acceleration of a human body; performing a massage operation; estimating activity information of the human body based on the measured acceleration; and controlling the massage operation based on the result of estimation.

In the massage program, the acceleration of the human body is measured, followed by estimation of the activity information of the human body based on the measured acceleration. Based on the result of estimation, the massage operation is controlled.

In this case, the massage operation is controlled based on the result of estimation of the activity information of the human body; so that it is possible to help recovery from fatigue more effectively in a short period of time according to the fatigue parts, working contents or degree of fatigue.

A physical activity determiner according to still another aspect of the present invention comprises: an acceleration measurement device that is attachable to a human body, and measures the acceleration of an attached part; and a determiner that determines activity information of the human body based on the acceleration measured by the acceleration measurement device.

In the physical activity determiner, the acceleration measurement device attachable to the human body measures the acceleration of its attached part, and the determiner determines the active information of the human body based on the measured acceleration. This allows for recognition of the fatigue parts of the human body, working contents or degree of fatigue.

The determiner may calculate a momentum variation based on the acceleration measured by the acceleration measurement device, to determine the active information of the human body based on the calculated momentum variation.

In this case, since a momentum variation is correlated with active information of the human body, it is possible to easily estimate the active information of the human body based on the momentum variation by measuring in advance the correlation therebetween.

The determiner may store in advance the relationship between momentum variations and active information of the human body, to determine the activity information of the human body based on the calculated momentum variation with reference to the stored relationship.

In this case, it is possible to easily determine the activity information of the human body based on the stored relationship between momentum variations and activity information.

A physical activity determination method according to still another aspect of the present invention comprises the steps of: measuring the acceleration of a human body, and determining the active information of the human body based on the measured acceleration.

In the physical activity determination method, the acceleration of the human body is measured, followed by determination of the active information of the human body based on the measured acceleration. This allows for recognition of the fatigue parts of the human body, working contents or degree of fatigue.

A computer-executable physical activity determination program according to still another aspect of the present invention makes the computer execute the processes of: obtaining acceleration from an acceleration measurement device that measures the acceleration of a human body; and determining active information of the human body based on the obtained acceleration.

In the physical activity determination program, the acceleration measurement device measures the acceleration of the human body, followed by determination of the activity information of the human body based on the measured acceleration. This allows for recognition of the fatigue parts of the human body, working contents or degree of fatigue.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a perspective view showing a relaxation system according to an embodiment of the present invention;

FIG. 2 is a block diagram showing an example of the relaxation system according to the present invention;

FIG. 3 is a schematic external view of the calorie meter;

FIG. 4 is a block diagram showing the inside structure of the main body of the calorie meter of FIG. 3;

FIG. 5 is a block diagram showing the inside structure of the massager;

FIG. 6 is an external perspective view of the massager;

FIG. 7 is a cross-section view of the massager;

FIG. 8 is an external perspective view of the remote controller of the massager;

FIG. 9 is a block diagram showing the inside structure of the air conditioner;

FIG. 10 is a block diagram showing the inside structure of the illuminator;

FIG. 11 is a block diagram showing the inside structure of the video/audio apparatus;

FIG. 12 is a flowchart showing an example of the operation of the CPU during the formation of parameters according to the acceleration detection program of the calorie meter;

FIG. 13 is a graph showing momentum variations ΔF per minute measured for a plurality of test subjects;

FIG. 14 is a flowchart showing the operation of the CPU according to an acceleration detection program during sampling of acceleration data;

FIG. 15 is a flowchart showing the operation of the control circuit of the massager;

FIG. 16 is a flowchart showing the operation of the control circuit of the massager;

FIG. 17 is a flowchart showing the operation of the control circuit of the massager;

FIG. 18(a) is a determination table that determines the condition of a human body based on the vital information;

FIG. 18(b) shows an example of a kneading pattern table;

FIG. 19 is a diagram for use in illustrating an example of the method of adjusting the data of the kneading pattern table selected by the control circuit;

FIG. 20(a) is an example of an air conditioning control table;

FIG. 20(b) is an example of an illumination adjustment control table;

FIG. 20(c) is an example of a video/audio adjustment control table;

FIG. 21 is a flowchart showing another example of the operation of the CPU according to the acceleration detection program during sampling of acceleration data;

FIG. 22 is a graph showing an example of the sum of engaged time for each of the ranges of the momentum variations ΔF calculated by the calorie meter;

FIG. 23 shows an example of an active body part determination table representing the relationship between each of the ranges of the momentum variations and the active part of body; and

FIG. 24 shows an example of a working activity determination table representing the relationship between each of the ranges of the momentum variations and the working activity.

### DESCRIPTION OF THE PREFERRED EMBODIMENTS

A relaxation system according to the present invention will hereinafter be described with reference to the drawings. In the embodiment, description is given with reference to a case where the relaxation system is applied to a massage chair (hereinafter referred to as a massager).

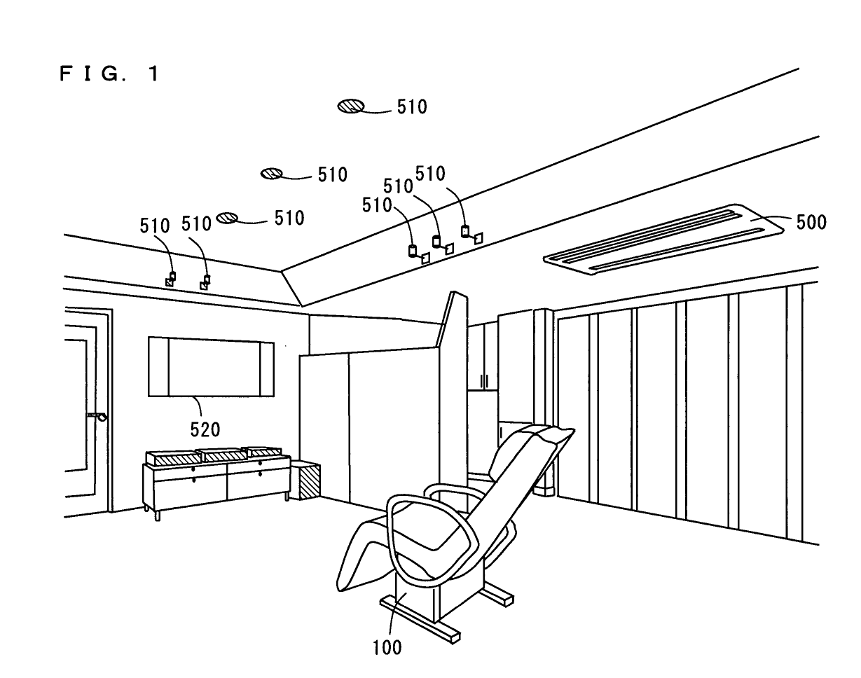

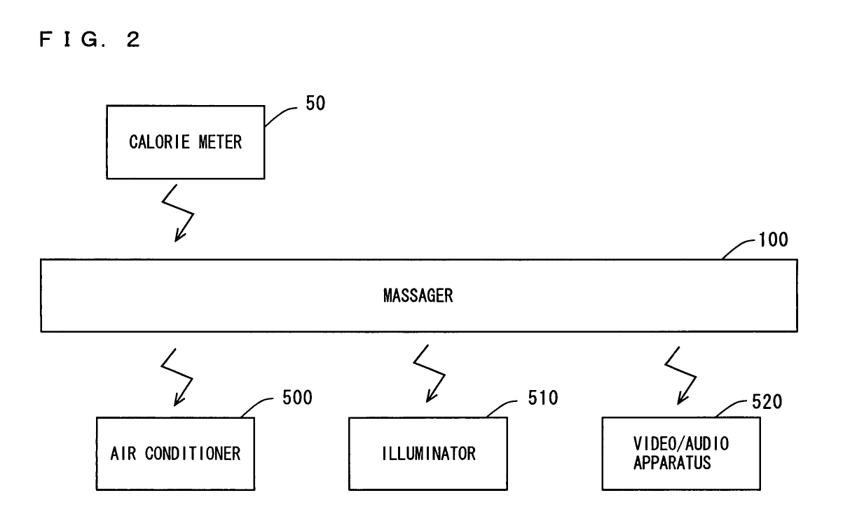

FIG. 1 is a perspective view showing a relaxation system according to an embodiment of the present invention. FIG. 2 is a block diagram showing en example of the relaxation system according to the present invention.

As shown in FIGS. 1 and 2, the relaxation system includes a calorie meter 50, a massager 100, an air conditioner 500, an illuminator 510, and a video/audio apparatus 520.

In the embodiment, the calorie meter 50 and the massager 100 constitute a massage system. The calorie meter 50 and the massager 100 also function as a physical activity determiner which determines the active part of a body, working activity, and amount of activity as active information of the body.

In the relaxation system shown in FIG. 1, the massager 100 is installed in a room, the video/audio apparatus 520 on the wall, and the air conditioner 500 and the illuminator 510 around the ceiling. The calorie meter 50 of FIG. 2 to be described later is attached around the waist of a user.

As shown in FIG. 2, the massager 100 receives momentum information from the calorie meter 50. The momentum information here refers to information on the amount of activity by a human body. This momentum information will later be described. The massager 100 controls the air conditioner 500, illuminator 510, and video/audio apparatus 520 based on the momentum information obtained from the calorie meter 50.

For a communication system in the embodiment between the calorie meter 50 and the massager 100, between the massager 100 and the air conditioner 500, between the massager 100 and the illuminator 510, or between the massager 100 and the video/audio apparatus 520, a radio communication system is employed. Examples of the radio communication systems include a microradio system(ex.Zigbee), specified low-power radio system, wireless LAN (Local Area Network), IrDA (Infrared Data Association), or any other wireless interface. When a wired communication system is employed rather than a radio communication system, a power line communication interface unit may for example be employed.

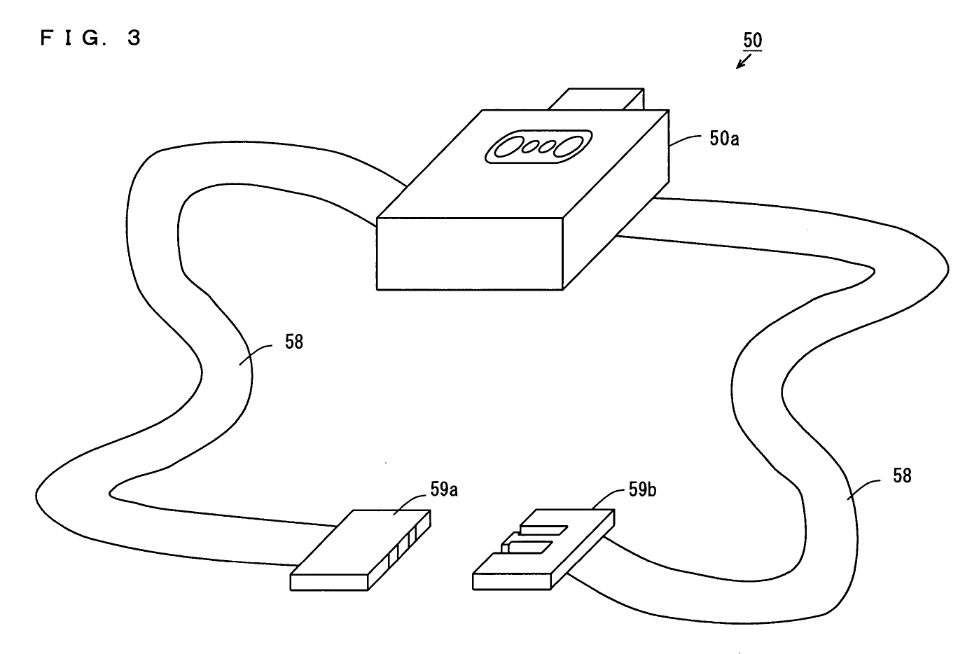

Now refer to FIG. 3 which is a schematic external view of the calorie meter 50.

As shown in FIG. 3, the calorie meter 50 includes a main body 50a, a belt 58, and buckles 59a, 59b. The calorie meter 50 is attachable to the user's waist by winding the belt 58 around his waist and fitting the buckles 59a, 59b with each other.

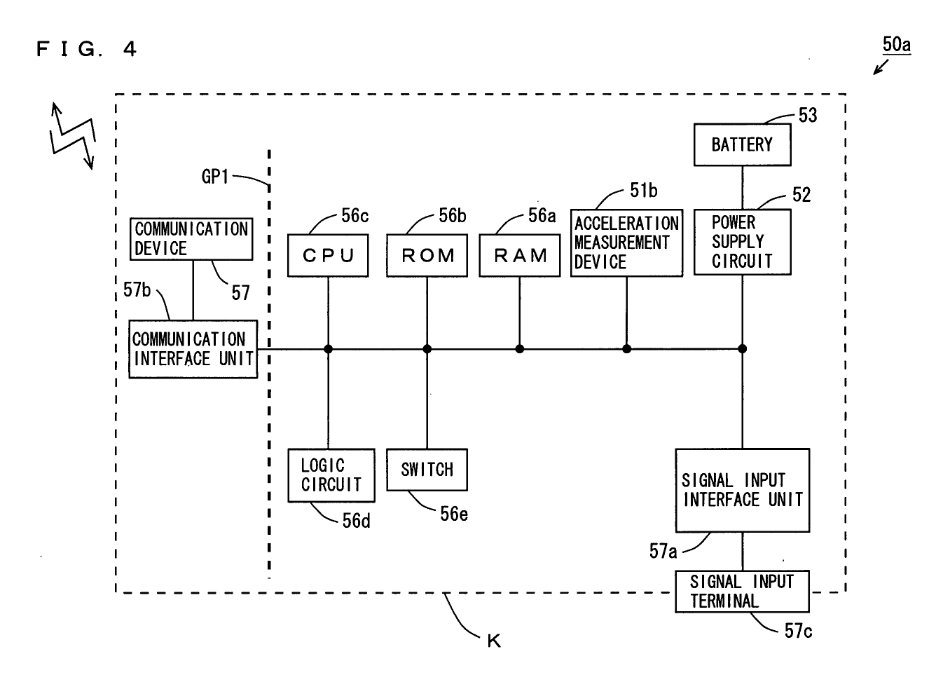

FIG. 4 is a block diagram showing the inside structure of the main body 50a of the calorie meter 50 of FIG. 3.

In FIG. 4, the main body 50a of the calorie meter 50 includes an acceleration measurement device 51b, a power supply circuit 52, a battery 53, a RAM (Random Access Memory) 56a, a ROM (Read Only Memory) 56b, a CPU (Central Processing Unit) 56c, a logic circuit 56d, a switch 56e, a communication device 57, a signal input interface 57a, a communication interface 57b, a signal input terminal 57c, and a casing K.

The communication device 57 and the communication interface unit 57b are separated from the other components through a ground plane GP1. The communication interface unit 57b, which is connected to the communication device 57, interconnects the CPU 56c and communication device 57. The communication device 57, employing the above-mentioned radio communication system, can be connected to the massager 100 of FIG. 2.

The ROM 56b stores a system program and an acceleration detection program. Other recording medium, such as a different memory, hard disc, magnetic disc, or optical disc, may instead be used as the recording medium that records the acceleration detection program. The RAM 56a stores acceleration data or the like to be described later. The CPU 56c executes the acceleration detection program stored in the ROM 56b on the RAM 56a. The acceleration detection program will later be described. The logic circuit 56d, including an analog-digital converter, a ring buffer, and the like, has its operation controlled by the CPU 56c.

The power supply circuit 52 connects the battery 53 and the other components inside the main body 50a of the calorie meter 50 to supply power of the battery 53 to each component. The signal input interface unit 57a interconnects the signal input terminal 57c and the CPU 56c, RAM 56a, and logic circuit 56d.

The switch 56e is connected to the CPU 56c to feed a given command signal to the CPU 56c based on the user manipulation.

The acceleration measurement device 51b is composed of acceleration sensors in three directions. The three acceleration sensors of the acceleration measurement device 51b measure accelerations in three axial directions in total, two of which being orthogonal to each other in a plane, and one of which vertical to the plane, to supply the measurements to the logic circuit 56d as acceleration data. The three axial directions as used herein are referred to as X direction, Y direction, and Z direction, respectively. Note that the acceleration measurement device 51b includes an analog-digital converter.

The casing K covers the entire components within the main body 50a of the calorie meter 50. Inside the casing K is present the one ground plane GP1 as described above. The casing K is thus divided into two spaces.

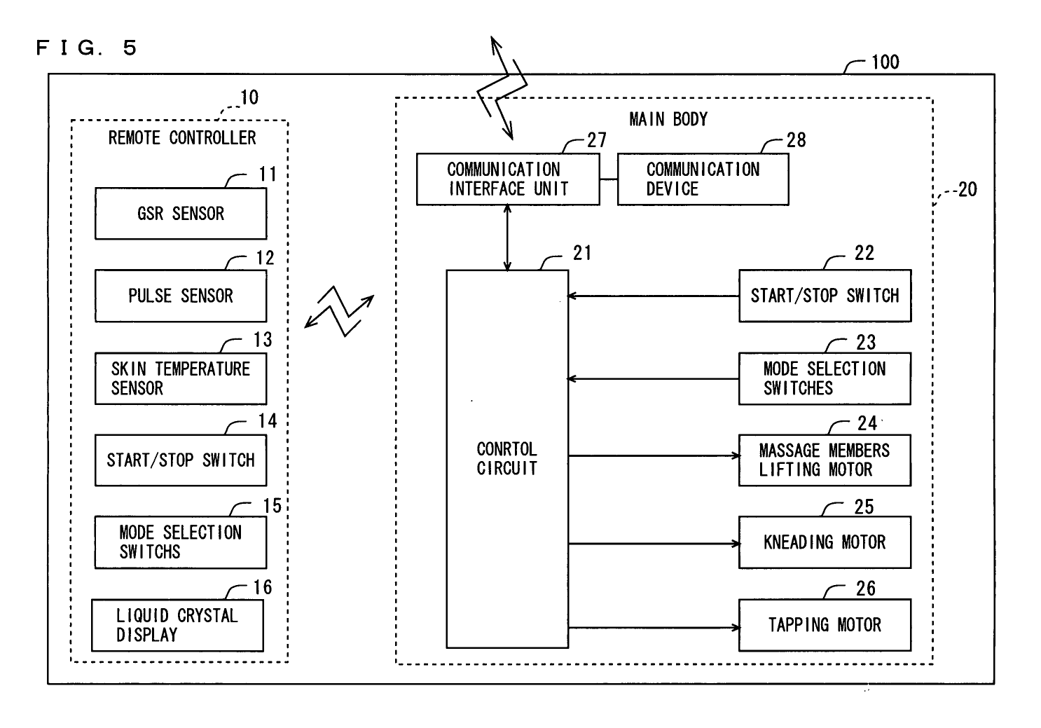

Now refer to FIG. 5 which is a block diagram showing the inside structure of the massager 100.

The massager 100 comprises a remote controller 10 and a main body 20.

The remote controller 10 principally includes a GSR (Galvanic Skin Response) sensor (hereinafter referred to as a GSR sensor) 11, a pulse sensor 12, a skin temperature sensor 13, a start/stop switch 14, a plurality of mode selection switches 15, and a liquid crystal display 16.

The main body 20 principally includes a control circuit 21, a start/stop switch 22, a plurality of mode selection switches 23, a massage members lifting motor 24, a kneading motor 25, a tapping motor 26, a communication interface unit 27, and a communication device 28.

The control circuit 21 includes a memory in which a relaxation control program is stored. The relaxation control program includes a massage control program for controlling the massager 100 based on momentum variations described later, and a relaxation apparatus control program for controlling the air conditioner 500, illuminator 510, and video/audio apparatus 520 based on momentum variations described later. This relaxation control program also includes an activity information determination program for determining, based on momentum variations described later, the active part, working activity, and amount of activity, as physical activity information.

The acceleration detection program and the massage control program stored in the ROM 56b of the calorie meter 50 constitute the massage program, the acceleration detection program and the relaxation control program constitute the relaxation program, and the acceleration detection program and the activity information determination program constitute the physical activity determination program.

Note that other recording medium, such as a different memory, hard disc, magnetic disc, or optical disc, may instead be used as the recording medium that records the relaxation control program.

Alternatively, all the relaxation program composed of the acceleration detection program and the relaxation control program may be stored in the ROM 56b of the calorie meter 50.

The remote controller 10 supplies vital information detected by the GSR sensor 11, pulse sensor 12, and skin temperature sensor 13 via the communication device 28 and communication interface unit 27 in the main body 20 to the control circuit 21. The vital information will later be detailed.

The control circuit 21 controls the operations of the massage members lifting motor 24, kneading motor 25, and tapping motor 26 in accordance with a massage program to be described later, and further controls the operations of the air conditioner 500, illuminator 510, and video/audio apparatus 520 of FIG. 2 via the communication interface unit 27.

With the start/stop switch 14 of the remote controller 10 or the start/stop switch 22 of the main body 20 being pressed, the control circuit 21 controls on/off for the massage members lifting motor 24, kneading motor 25, and tapping motor 26. Also, with the mode selection switches 15 of the remote controller 10 or the mode selection switches 23 of the main body 20 being pressed, the control circuit 21 alters either operation of the massage members lifting motor 24, kneading motor 25, or tapping motor 26. When, for example, the kneading motor 25 operation has been started while the tapping motor 26 operation is being ceased, pressing the mode selection switches 23 will cause the kneading motor 25 operation to be ceased, and the tapping motor 26 operation be started.

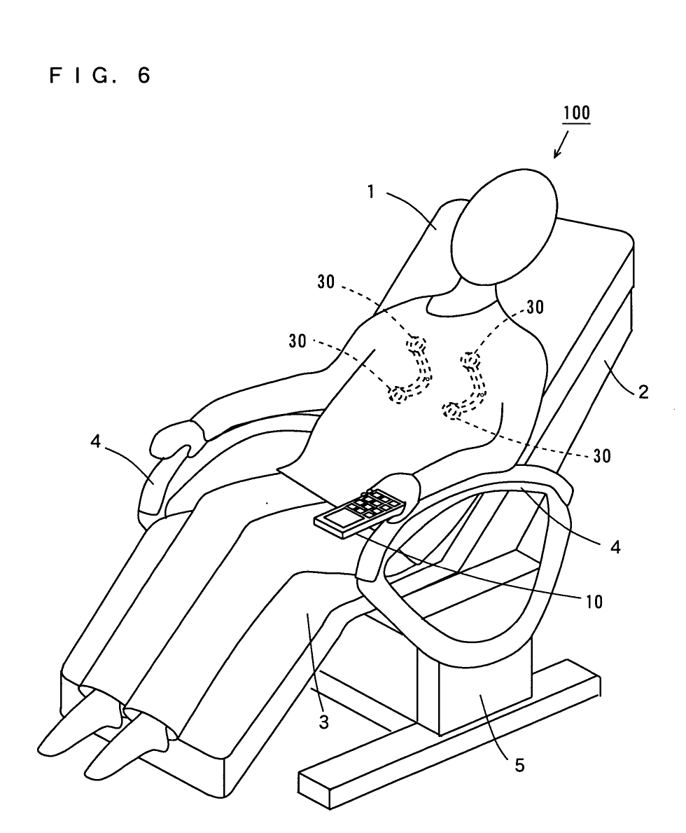

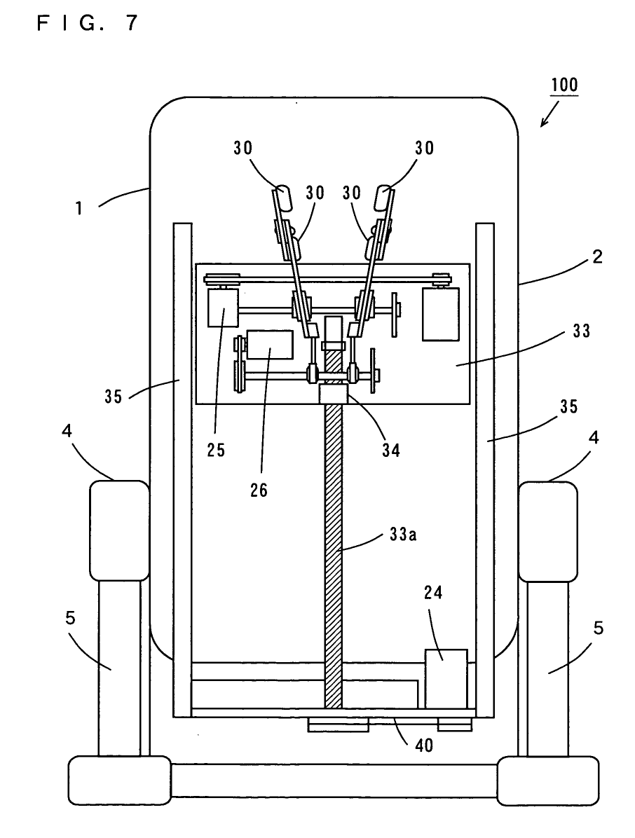

Now, mechanism portion of the massager 100 will be described. FIG. 6 is an external perspective view of the massager 100, and FIG. 7 is a cross-section view of the massager 100.

The massager 100 shown in FIGS. 6 and 7 principally comprises a backrest 1, a massage mechanism 2, a seat 3, a pair of left and right armrests 4, and legs 5. Inside the massage mechanism 2 are provided with a plurality of massage members 30.

As shown in FIG. 7, the massage mechanism 2 is provided with a massage members drive 33. The massage members drive 33 is upwardly/downwardly movably supported along a pair of side frames 35. Below the massage members drive 33 is provided a massage members lifting motor 24, of which the driving force is transmitted via a driving force transmission mechanism 40 of the belt type to a ball screw 33a.

The ball screw 33a is screwed on a bearing 34 provided in the massage members drive 33. The massage members 30 also have a plurality of link mechanisms. The driving forces of the kneading motor 25 and the tapping motor 26 are transmitted through the plurality of link mechanisms to the massage members 30.

As a result of this, the ball screw 33 is rotated by a massage members lifting motor 24, causing the massage members drive 33 to move upward or downward. Then, the massage members 30 are operated by the kneading motor 25 and tapping motor 26 in such a way as to press the rear side of the human body.

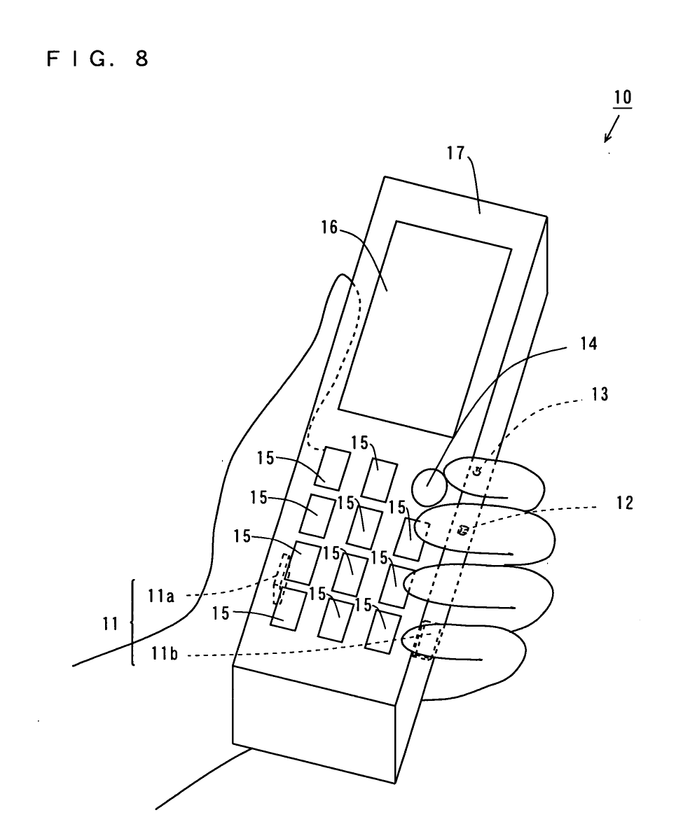

Now refer to FIG. 8 which is an external perspective view of the remote controller 10 of the massager 100.

As already mentioned, the inside structure of the remote controller, which is housed in the casing 17, comprises the GSR sensor 11, pulse sensor 12, skin temperature sensor 13, start/stop switch 14, plurality of mode selection switches 15, and liquid crystal display 16.

On one face of the upright casing 17 are provided the start/stop switch 14, plurality of mode selection switches 15, and liquid crystal display 16. On one side face of the casing 17 are provided the pulse sensor 12 composed of a light emitting device and a light receiving device, and the skin temperature sensor 13 composed of a thermistor. In addition, on the one side face and the opposite other side face are provided a pair of electrodes 11a, 11b, respectively, constituting the GSR sensor 11.

The user grasps this remote controller 10 by the hand. In this case, the index finger comes into contact with the skin temperature sensor 13, the middle finger with the pulse sensor 12, the ring finger and little finger with the one electrode 11b of the GSR sensor 11, and the base of the thumb or palm of the hand with the other electrode 11a of the GSR sensor 11.

The liquid crystal display 16 of the remote controller 10 displays the part being massaged, the degree of stiffness, the degree of comfortableness, the position of a stiff part, and the like.

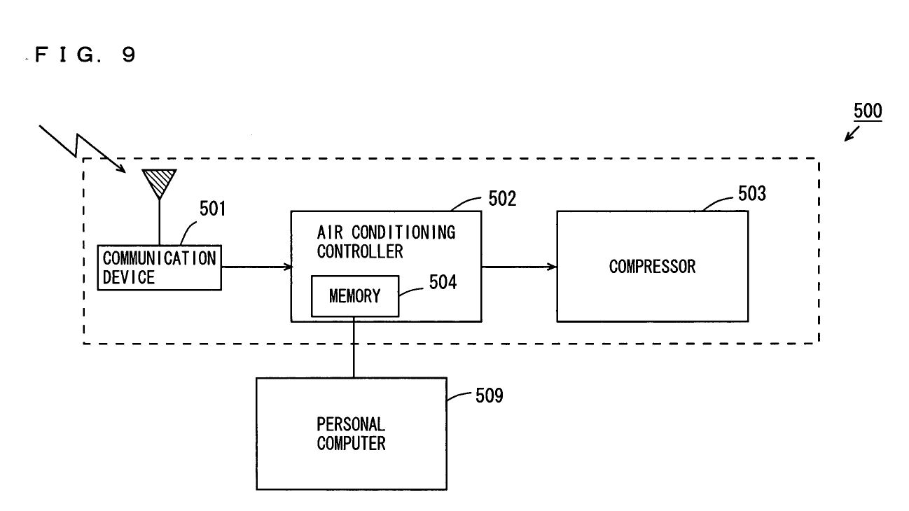

Now refer to FIG. 9 which is a block diagram showing the inside structure of the air conditioner 500.

As shown in FIG. 9, the air conditioner 500 includes a communication device 501, an air conditioning controller 502, and a compressor 503. The air conditioning controller 502 includes a memory 504. Further, the air conditioner 500 can be connected to a personal computer 509.

An air conditioning control table for controlling the operation of the compressor 503 is created by manipulating the personal computer 509. This air conditioning control table will later be detailed. The created air conditioning control table is stored within the memory 504.

The air conditioning controller 502 reads the air conditioning control table stored within the memory 504 in response to the signal fed from the massager 100 via the communication device 501, to control the operation of the compressor 503 based on the air conditioning control table.

FIG. 10 is a block diagram showing the inside structure of the illuminator 510.

As shown in FIG. 10, the illuminator 510 includes a communication device 511, an illumination controller 512, and a LED (Light Emitting Diode) illumination 513. The LED illumination in general is capable of expressing colors as many as 16,770,000. The illumination controller 512 includes a memory 514. Further, the illumination controller 512 can be connected with a personal computer 519.

An illumination adjustment control table for controlling the operation of the LED illumination 513 is created by manipulating the personal computer 519. This illumination adjustment control table will later be detailed. The created illumination adjustment control table is stored within the memory 514.

The illumination controller 512 reads the illumination adjustment control table stored with in the memory 514 in response to the signal fed from the massager 100 via the communication device 511, to control the operation of the LED illumination 513 based on the illumination adjustment control table.

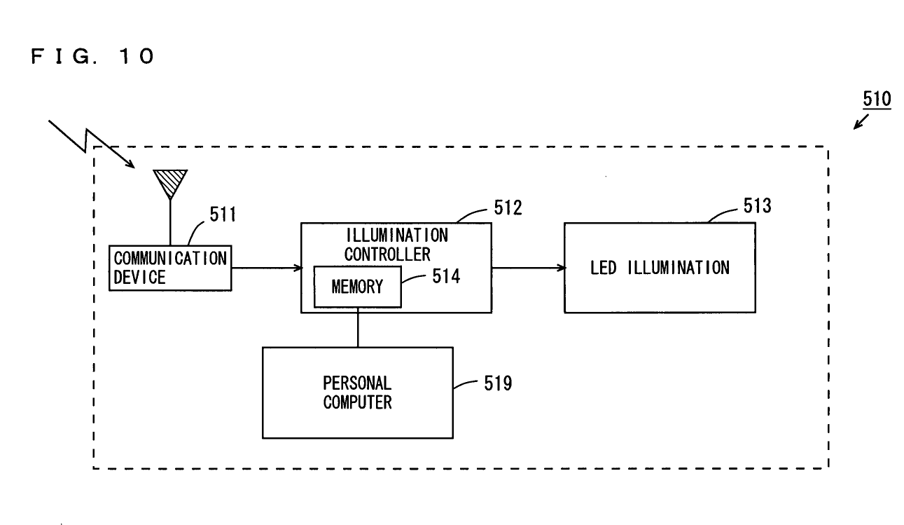

FIG. 11 is a block diagram showing the inside structure of the video/audio apparatus 520.

As shown in FIG. 11, the video/audio apparatus 520 includes a communication device 521, a video/audio controller 522, a display 523, and a speaker 525. The video/audio controller 522 includes a memory 524. Further, the video/audio controller 522 can be connected with a personal computer 529.

A video/audio adjustment control table for controlling the operations of the display 523 and the speaker 525 is created by manipulating the personal computer 529. This video/audio adjustment control table will be detailed later. The created video/audio adjustment control table is stored within the memory 524.

The video/audio controller 522 reads the video/audio adjustment control table stored within the memory 524 in response to the signal fed from the massager 100 via the communication device 521, to control the operations of the display 523 and the speaker 525 based on the video/audio adjustment control table.

Now, the acceleration detection program recorded in the ROM 56b of the calorie meter 50 will be described.

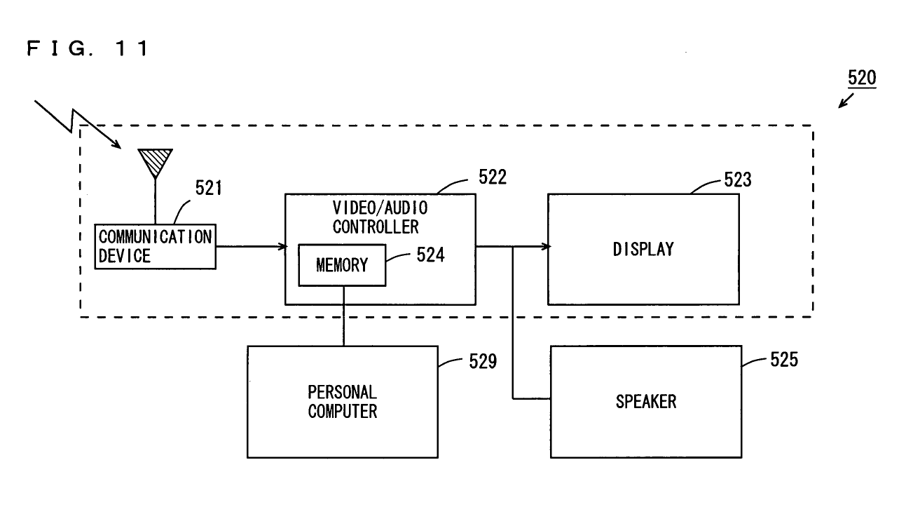

FIG. 12 is a flowchart showing an example of the operation of the CPU 56c during the formation of parameters according to the acceleration detection program of the calorie meter 50. The formation of parameters shown in FIG. 12 is performed during the design or manufacture of the relaxation system.

Initially, a test subject is made to wear a mask for a portable indirect calorimetry system (not shown), and also wear the calorie meter 50 shown in FIG. 3 around his waist before conducting various kinds of exercises. The various kinds of exercises here refer to a kneeling posture, an upright posture, bending and stretching exercises, walking (4 km/h), quick walking (6 km/h), arm exercises with a load (dumbbell) on the hand (hereinafter referred to as dumbbell exercises), and exercises using the overall body with a load (dumbbell) on the hand (hereinafter simply referred to as overall body exercises).

The CPU 56c of the calorie meter 50 samples output from the portable indirect calorimetry system (Step S1). The sampling data from the portable indirect calorimetry system means the energy expenditure of the test subject.

Following this, the CPU 56c samples acceleration data from the acceleration measurement device 51b of the calorie meter 50 (Step S2).

Note that the acceleration data here means a plurality of pieces of acceleration data corresponding to the X, Y, and Z directions, because the acceleration measurement device 51b is composed of the acceleration sensors in the three directions, as mentioned above.

Then, the CPU 56c removes gravity components from the sampled pieces of acceleration data (Step S3). Note that owing to the constant gravitational influence on the acceleration measurement device 51b, the sampled pieces of acceleration data always contain gravitational acceleration data, even if the test subject is not conducting the various kinds of exercises mentioned above. Thus, in order to set the value when the test subject is not conducting exercises as a reference (0), the gravity components are removed from the sampled pieces of acceleration data, using a high-pass filter passing a frequency of 1 Hz or greater. This results in information representing an actual amount of exercises conducted by the test subject.

Then, the CPU 56c calculates acceleration indices (Step S4). Acceleration indices Acci of the calorie meter 50 representing the momentum information for the test subject is calculated according to the equation below:
Acci=(\sqrt{Xi²+Yi²+Zi²})×W/Fs
where Fs represents the sampling cycle of the acceleration data, W represents the weight of the test subject, Xi represents the sampling result of the ith acceleration data in the X direction, Yi represents the sampling result of the ith acceleration data in the Y direction, and Zi represents the sampling result of the ith acceleration data in the Z direction.

According to the above equation, based on the acceleration data of the test subject from which the gravity components in the X, Y, and Z directions are removed, the magnitude of the acceleration is found, the found magnitude of acceleration is multiplied by the weight W of the test subject, and the product is divided by the sampling cycle Fs. In the embodiment, the sampling cycle Fs is 25 Hz.

The CPU 56c subsequently determines whether or not the acceleration data per second is available (Step S5). For example in the embodiment, with the sampling cycle Fs of 25 Hz, the CPU 56c determines whether or not twenty-five pieces of acceleration data have been sampled. Upon determining that there is no acceleration data per second available, the CPU 56c returns to Step S1 to repeat the processes of Step S1 through S5.

On the other hand, upon determining that there is the acceleration data per second available, the CPU 56c calculates a momentum variation per minute (Step S6). A momentum variation per minute can be expressed by the equation below:
ΔF=60×εAcci/W
where ΔF represents a momentum variation per minute. According to the above equation, the momentum variation ΔF per minute is calculated by multiplying by 60 the sum of the acceleration indices Acci per minute, and dividing the product by the weight W of the test subject. Measurements of momentum variations ΔF are conducted for a plurality of test subjects.

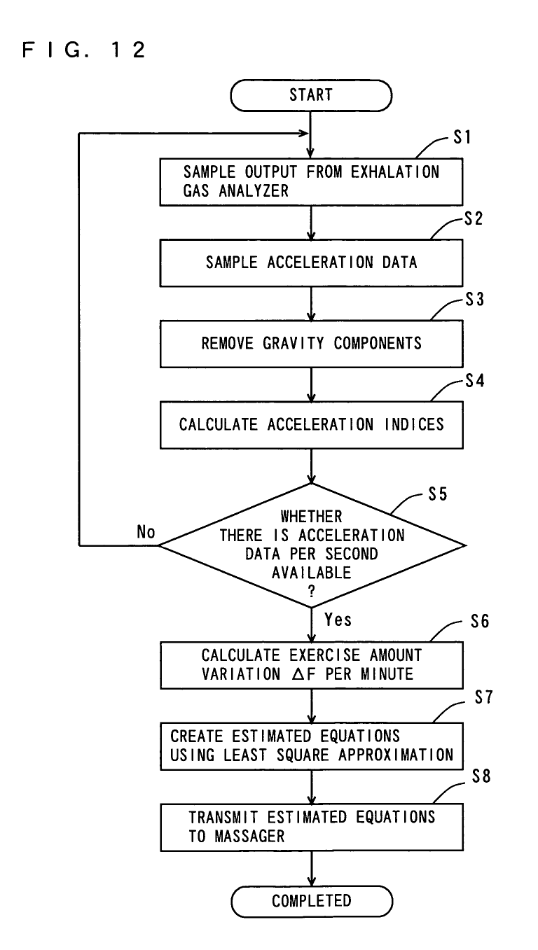

Now, the momentum variations ΔF measured from the plurality of test subjects will be described. FIG. 13 is a graph showing the momentum variations ΔF per minute measured for the plurality of test subjects.

In FIG. 13, the ordinate shows the energy expenditure (Kcal/min kg), the abscissa shows the momentum variation ΔF.

In FIG. 13, the momentum variations ΔF per minute indicate the results of which the plurality of test subjects are made to wear masks for the portable indirect calorimetry system (not shown) with the calorie meters 50 shown in FIG. 3 around their waist, to conduct kneeling posture, upright posture, bending and stretching exercises, walking, quick walking, dumbbell exercises, and overall body exercises.

In FIG. 13, the range a shows momentum variations ΔF for the kneeling and upright postures, the range b shows momentum variations ΔF for the dumbbell exercises, the range c shows momentum variations ΔF for the bending and stretching exercises, the range d shows momentum variations ΔF for the overall body exercises, the range e shows momentum variations ΔF in walking, and the range f shows momentum variations ΔF in quick walking.

It can be seen from FIG. 13 that for the exercises not involving the movement of the test subjects, the values of the momentum variations ΔF are less than 9, whereas for the exercises involving the movement, the values of the momentum variations ΔF are not less than 9.

In this case, the value of 9 for the momentum variation ΔF serves as the threshold for determining the working activity. That is, in an activity determination process described later, when the value of the momentum variation ΔF is less than 9, the working activity can be determined as the exercise not involving the movement of the user, whereas when the value is not less than 9, the working activity can be determined as the exercise involving the movement of the user.

The CPU 56c subsequently creates estimated equations using the least square approximation based on the results of momentum variations ΔF as shown in FIG. 13 (Step S7 of FIG. 12). For example, one estimated equation is created for the ranges a to d of FIG. 13 with the least square approximation, and another estimated equation is created for the ranges e and f, with the least square approximation.

Then, the CPU 56c transmits the two created estimated equations to the massager 100 (Step S8). Thus, the operation of the CPU 56c during the formation of parameters according to the acceleration detection program of the calorie meter 50 is completed.

In the embodiment, the created estimated equations are transmitted to the massager 100 by the CPU 56c; note, however, that the created estimated equations may be stored in the RAM 56a by the CPU 56c. In that case, the CPU 56c is capable of determining the active part of body, working activity, and amount of activity as will be described later, using the estimated equations stored in the RAM 56a.

In addition, in the embodiment, the momentum variation ΔF per minute is calculated by multiplying by 60 the sum of the acceleration indices Acci per second; note, however, that the momentum variation ΔF per minute may be calculated based on the pieces of acceleration data obtained from one-minute long sampling.

FIG. 14 is a flowchart showing the operation of the CPU 56c according to the acceleration detection program during sampling of the acceleration data. The user leads every day life with the calorie meter 50 put on.

Initially, the CPU 56c of the calorie meter 50 samples the acceleration data from the acceleration measurement device 51b of the calorie meter 50 (Step S11). In this case, the user is not wearing the portable indirect calorimetry system.

Then, the CPU 56c removes gravity components from the plurality of pieces of acceleration data (Step S12). The process of removing the gravity components from the pieces of acceleration data is similar to that as described above. Following this, the CPU 56c calculates acceleration indices Acci (Step S13).

Then, the CPU 56c determines whether or not the acceleration data per second is available (Step S14). With the sampling cycle Fs of 25 Hz, for example, it determines whether or not twenty-five pieces of acceleration data have been sampled. Upon determining that there is no acceleration data per second available, the CPU 56c returns to Step S11 to repeat the processes of Step S11 through S14.

On the other hand, upon determining that there is the acceleration data per second available, the CPU 56c stores the acceleration indices Acci into the RAM 56a (Step S15). The CPU 56c calculates acceleration indices Acci for each minute for storage into the RAM 56a. Subsequently, the CPU 56c determines whether or not the user has started using the massager 100 (Step S16). When the CPU 56c determines at this point that the massager 100 has not been used, it returns to Step S11 to repeat the processes of Step S11 through S14 for each minute.

On the other hand, upon determining that the user has started using the massager 100, the CPU 56c transmits the acceleration indices Acci stored in the RAM 56a to the massager 100 (Step S17). Thus, the operation of the CPU 56c according to the acceleration detection program is completed extracting the acceleration indices Acci based on the actual acceleration data from the user wearing the calorie meter 50.

In the embodiment, the acceleration indices Acci are transmitted to the massager 100 by the CPU 56c; note, however, that the CPU 56c may determine the active part of body, working activity, and amount of activity described later based on the acceleration indices Acci stored in the RAM 56a.

In addition, in the embodiment, it is determined whether or not the acceleration data per second is available at Step S14; note, however, that determination may be made as to whether or not there is acceleration data per minute available.

Description is now made of the control circuit 21 that operates based on the acceleration indices Acci transmitted according to the above acceleration detection program.

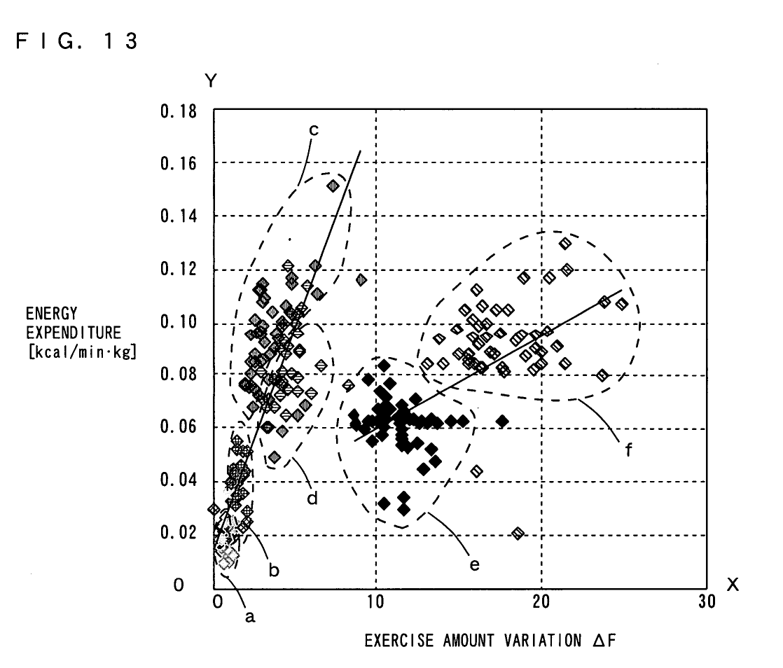

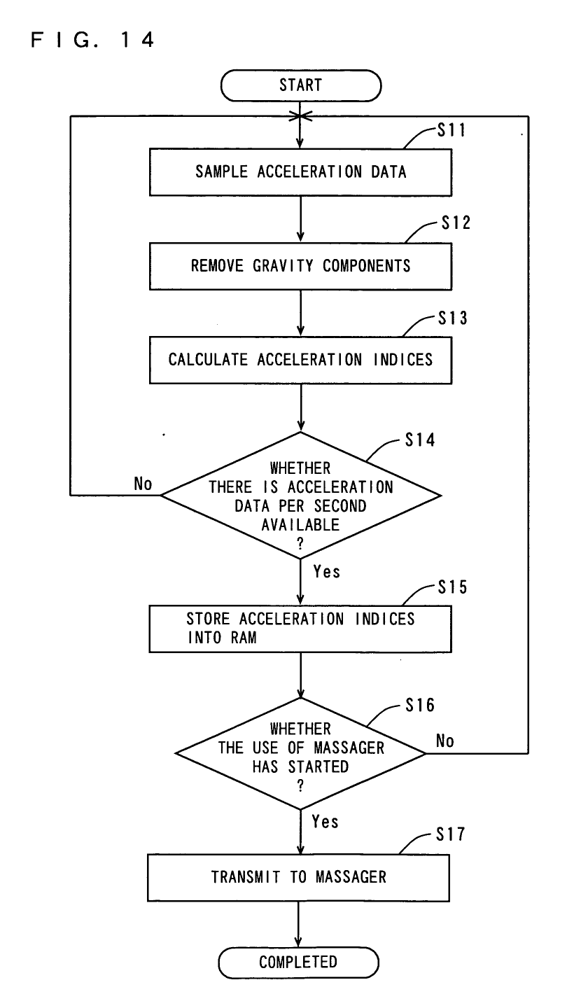

FIGS. 15, 16, and 17 are flowcharts showing the operation of the control circuit 21 of the massager 100 according to the relaxation control program.

Initially, the control circuit 21 of the massager 100 receives a test kneading signal (Step S21). In the embodiment, by manipulating the mode selection switches 15 on the remote controller 10 shown in FIG. 5, the test kneading signal is transmitted to the control circuit 21 of the massager 100.

Next, the control circuit 21 of the massager 100 instructs the kneading motor 25 and massage members lifting motor 24 to start running (Step S22). This causes the massage members 30 of FIG. 6 to start upward/downward movements along the rear side of the human body.

While the massage members 30 are moving upwardly/downwardly along the rear side of the human body, the control circuit 21 of the massager 100 subsequently samples vital information detected by the GSR sensor 11 (Step S23). The vital information as used herein represents information varying according to the degree of relaxation (degree of stress relief) or degree of stress. This vital information shows a low value of activity when the human body is in a relaxed state, while showing a high value of activity when the human body is in a state of tension. Therefore, when the vital information detected by the GSR sensor 11 during the test kneading shows a high value, it is estimated that there is a feeling of stiffness, whereas when the vital information shows a low value, there is no feeling of stiffness.

After this, the control circuit 21 determines whether or not the vital information supplied from the GSR sensor 11 is abnormal (Step S24). Abnormalities in the vital information may occur when, for example, the user is not grasping the remote controller 10 as shown in FIG. 8, with his hand failing to contact the GSR sensor 11, which prevents the detection of any vital information.

Upon determining that the vital information from the GSR sensor 11 is abnormal, the control circuit 21 makes the liquid crystal display 16 of the remote controller 10 provide a display informing the abnormality (Step S25). This allows the user to see the display on the liquid crystal display 16 to appropriately grasp the remote controller 10.

Then, the control circuit 21 returns to the process of Step S23 to repeat the sampling of vital information from the GSR sensor 11.

On the other hand, upon determining that the vital information detected from the GSR sensor 11 is normal, the control circuit 21 performs noise processing for the detected vital information (Step S26).

The control circuit 21 subsequently detects a variation in the vital information from the GSR sensor 11 for each of phase sections (Step S27). The term phase sections as used herein refers to the sections of the range in which the massage members 30 are moved upward and downward by the massage members lifting motor 24 that are divided at given spacings. More specifically, the sections of the range of the upward and downward movements of the massage members 30 divided into the corresponding sections of the neck, shoulders, back, and waist of the rear side of the human body.

After this, while the massage members 30 are moving upward and downward along the rear side of the human body, the control circuit 21 of the massager 100 samples vital information detected from the pulse sensor 12 (Step S28). The vital information here is similar to that described above.

The control circuit 21 subsequently determines whether or not the vital information supplied from the pulse sensor 12 is abnormal (Step S29). Abnormalities in the vital information may occur when, for example, the user is not grasping the remote controller 10 as shown in FIG. 8, with his hand failing to contact the pulse sensor 12, which prevents the detection of any vital information.

Upon determining that the vital information supplied from the pulse sensor 12 is abnormal, the control circuit 21 makes the liquid crystal display 16 of the remote controller 10 provide a display informing the abnormality (Step S30). This allows the user to see the display on the liquid crystal display 16 to appropriately grasp the remote controller 10.

After that, the control circuit 21 returns to the process of Step S28 to repeat the sampling of vital information from the pulse sensor 12.

On the other hand, upon determining that the vital information detected from the pulse sensor 12 is normal, the control circuit 21 performs noise processing for the detected vital information (Step S31).

Subsequently, the control circuit 21 detects the pulse rate based on the noise processed vital information (Step S32).

Following this, the control circuit 21 detects a variation in the vital information from the pulse sensor 12 for each of the phase sections.

While the massage members 30 are moving upward and downward along the rear side of the human body, the control circuit 21 of the massager 100 subsequently samples vital information detected by the skin temperature sensor 13 (Step S34). The vital information is similar to that described above.

The control circuit 21 then determines whether or not the vital information supplied from the skin temperature sensor 13 is abnormal (Step S35). Abnormalities in the vital information may occur when, for example, the user is not grasping the remote controller 10 as shown in FIG. 8, with his hand failing to contact the skin temperature sensor 13, which prevents the detection of any vital information.

Upon determining that the vital information supplied from the skin temperature sensor 13 is abnormal, the control circuit 21 makes the liquid crystal display 16 of the remote controller 10 provide a display informing the abnormality (Step S36). This allows the user to see the display on the liquid crystal display 16 to appropriately grasp the remote controller 10.

After that, the control circuit 21 returns to the process of Step S34 to repeat the sampling of vital information from the skin temperature sensor 13.

On the other hand, upon determining that the vital information detected from the skin temperature sensor 13 is normal, the control circuit 21 performs noise processing for the detected vital information (Step S37).

After this, the control circuit 21 detects a variation in the vital information from the skin temperature sensor 13 for each of the phase sections (Step S38).

Variations in the vital information are detected by the foregoing operation of the control circuit 21. Following is a description of the operation of the control circuit 21 using the detected variations in the vital information.

The control circuit 21 reads a determination table and a kneading pattern table already stored in the storage device (not shown) incorporated therein (Step S39). The determination table and the kneading pattern table will later be described.

The control circuit 21 then selects an item from the determination table based on the vital information sampled from the GSR sensor 11, pulse sensor 12, and skin temperature sensor 13, followed by selection of an item from the kneading pattern table based on the determination table (Step S40).

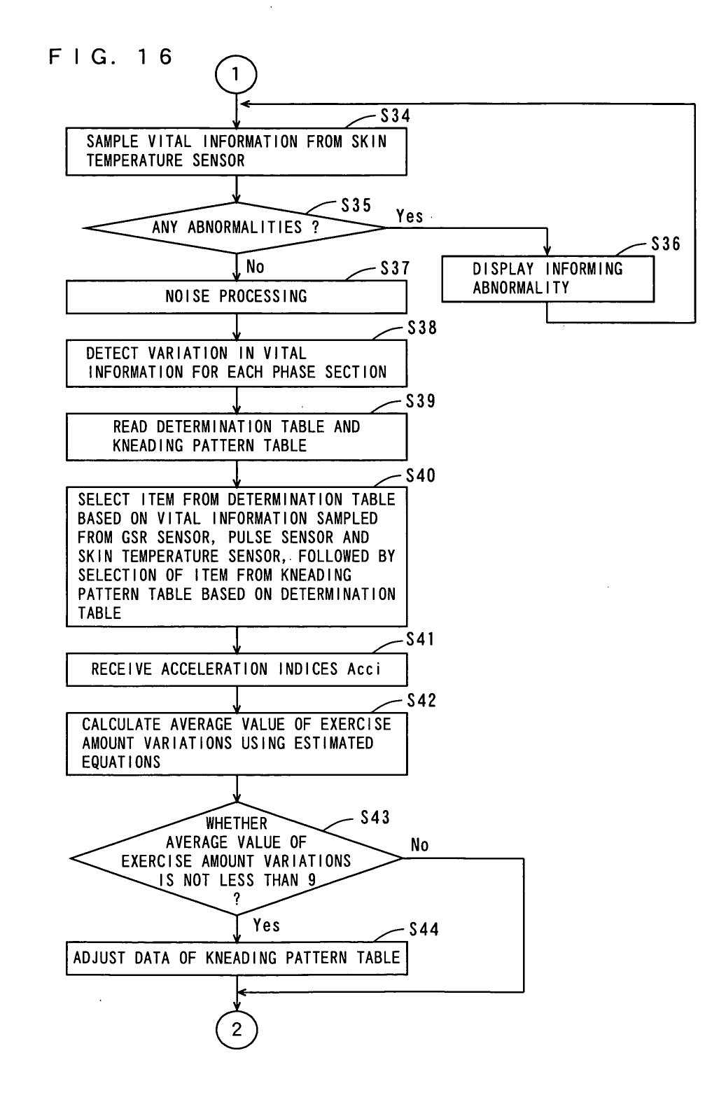

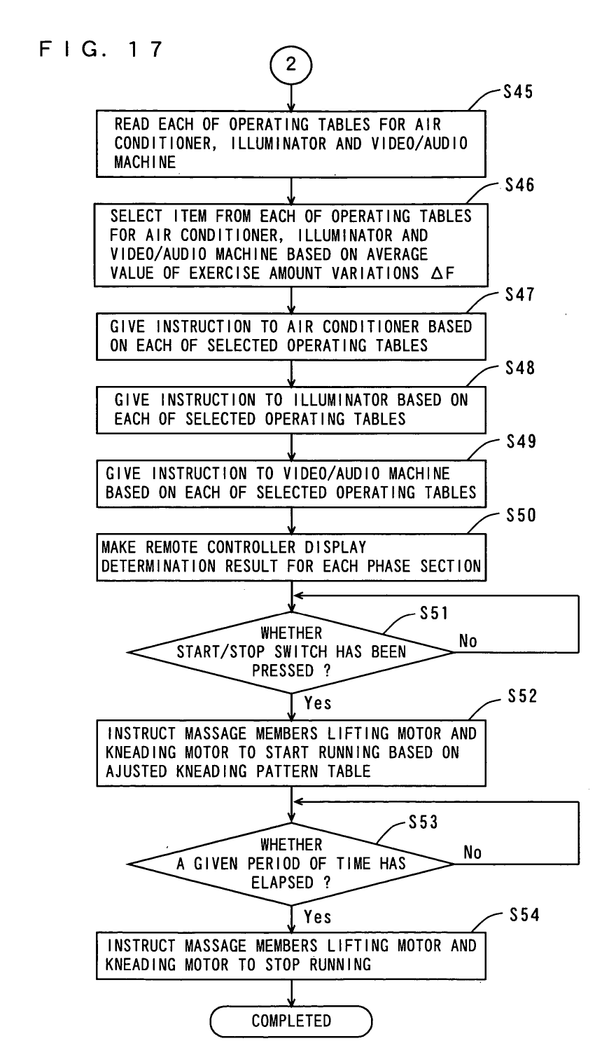

FIG. 18(a) shows the determination table for determining the condition of the human body based on the vital information sampled from the GSR sensor 11, pulse sensor 12, and skin temperature sensor 13. FIG. 18(b) shows an example of the kneading pattern table for which selection is done based on the determination table.

As shown in FIG. 18(a), the control circuit 21 makes an assessment as to whether the user is “relaxed”, “neutral”, “active” or “feeling pain” based on the vital information from the GSR sensor 11, pulse sensor 12, and skin temperature sensor 13. The control circuit 21 selects an item from the kneading pattern table shown in FIG. 18(b) for each of the phase sections according to the determination result.

After this, the control circuit 21 receives the acceleration indices Acci obtained based on the acceleration data transmitted from the remote controller 10 (Step S41). Note that the acceleration indices Acci, which are calculated for each minute, include multiple pieces of data.

The control circuit 21 calculates momentum variations ΔF for the human body, using each of the acceleration indices Acci and the two estimated equations previously supplied from the remote controller 10 (Step S42).

The control circuit 21 subsequently determines whether or not the average value of the calculated momentum variations ΔF is not less than 9 (Step S43). Upon determining that the average value of the momentum variations ΔF is less than 9, the control circuit 21 moves on to the process of Step S45.

On the other hand, upon determining that the average value of the momentum variations ΔF is not less than 9, the control circuit 21 makes an adjustment to the data of the kneading pattern table selected in the process of Step S40 (Step S44). In the embodiment, determination is made based on the average value of the momentum variations ΔF; note, however, determination may be made as to whether or not the momentum variations ΔF are not less than 9 for an hour or longer. Alternatively, determination may be made using any other value of the momentum variation ΔF.

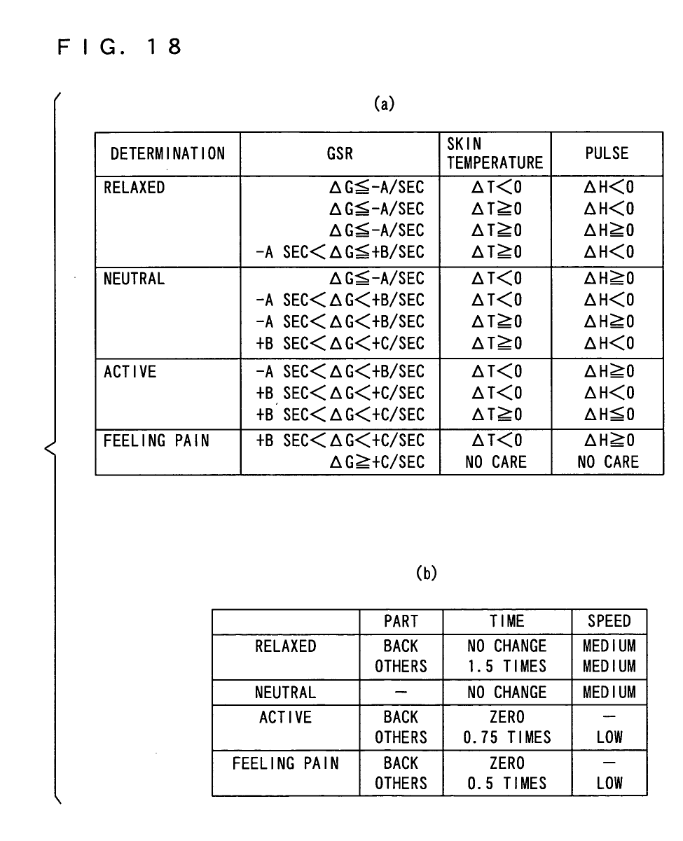

FIG. 19 is a diagram for use in illustrating the method of adjusting the data of the kneading pattern table selected by the control circuit 21.

As shown in FIG. 19, when the average value of momentum variations ΔF is less than 9, it is assumed as explained above that the legs of the human body are not being used, so that the operating speed and time for leg massage are adjusted to half. When, on the other hand, the average value of momentum variations ΔF is not less than 9 and less than 20, the operating speed and time are adjusted to once. In the embodiment, determination is not made as to whether or not the average value of the momentum variations ΔF is not less than 20 or 30. In the case of such determinations, when the average value of the momentum variations ΔF is determined as not less than 20 and less than 30, the operating speed and time are adjusted to 1.5 times. When the average value of the momentum variations ΔF is determined as not less than 30, the operating speed is adjusted to 1.5 times and the operating time to twice.

The control circuit subsequently reads each of operating tables for the air conditioner 500, illuminator 510, and video/audio apparatus 520 (Step S45). Each of the operating tables will later be detailed.

Based on the average value of the momentum variations ΔF, the control circuit 21 subsequently selects an item from each of the operating tables for the air conditioner 500, illuminator 510, and video/audio apparatus 520 (Step S46).

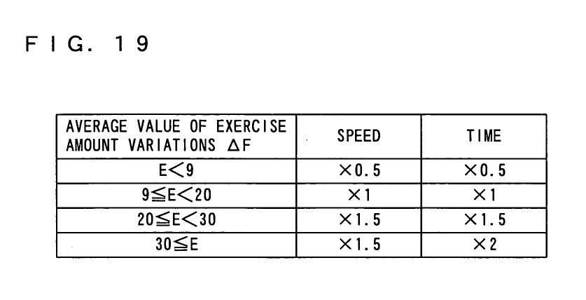

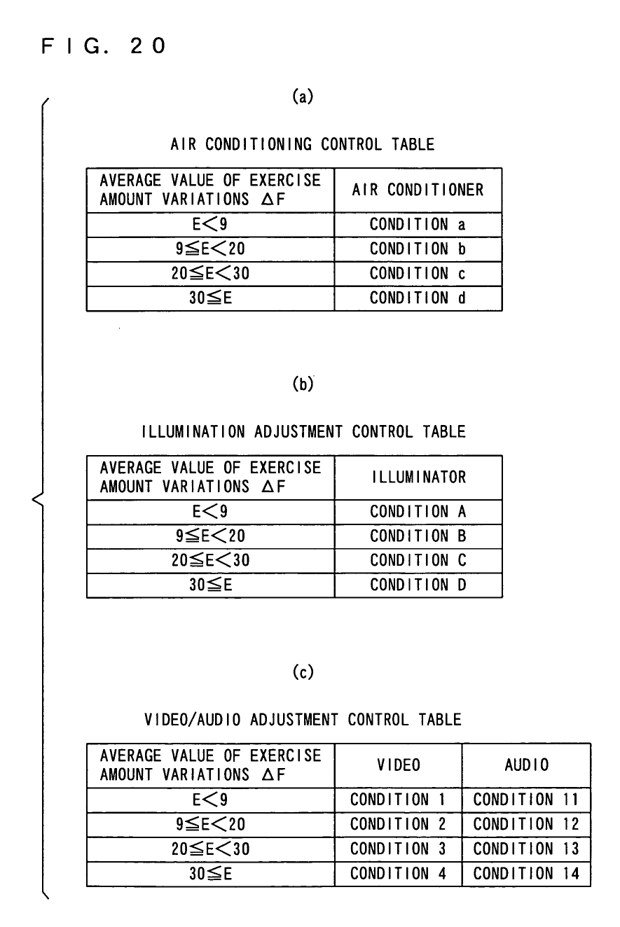

Each of the operating tables is now explained. FIG. 20(a) shows an example of an air conditioner control table stored within the memory 504 of the air conditioner 500; FIG. 20(b) shows an example of an illumination adjustment control table stored within the memory 514 of the illuminator 510; and FIG. 20(c) shows an example of a video/audio adjustment control table stored within the memory 524 of the video/audio apparatus 520.

For example, referring to FIG. 20(a), the air conditioning control table shows that the air conditioning controller 502 of FIG. 9 controls the operation of the compressor 503 under the condition a, when the average value of the momentum variations ΔF is less than 9; under the condition b, when the average value of the momentum variations ΔF is not less than 9 and less than 20; under the condition c, when the average value of the momentum variations ΔF is not less than 20 and less than 30; and under the condition d, when the average value of the momentum variations ΔF is not less than 30.

Referring to FIG. 20(b), the illumination adjustment control table shows that the illumination controller 512 of FIG. 10 controls the operation of the LED illumination 513 under the condition A, when the average value of the momentum variations ΔF is less than 9; under the condition B, when the average value of the momentum variations ΔF is not less than 9 and less than 20; under the condition C, when the average value of the momentum variations ΔF is not less than 20 and less than 30; and under the condition D, when the average value of the momentum variations ΔF is not less than 30.

Referring to FIG. 20(c), the video/audio adjustment control table shows that when the average value of the momentum variations ΔF is less than 9, the video/audio controller 522 of FIG. 11 controls the operation of the display 523 under the condition 1, while controlling the operation of the speaker 525 under the condition 11; when the average value of the momentum variations ΔF is not less than 9 and less than 20, the video/audio controller 522 controls the operation of the display 523 under the condition 2, while controlling the operation of the speaker 525 under the condition 12; when the average value of the momentum variations ΔF is not less 20 and less than 30, the video/audio controller 522 controls the operation of the display 523 under the condition 3, while controlling the operation of the speaker 525 under the condition 13; and when the average value of the momentum variations ΔF is not less than 30, the video/audio controller 522 controls the operation of the display 523 under the condition 4 while controlling the speaker 525 under the condition 14.

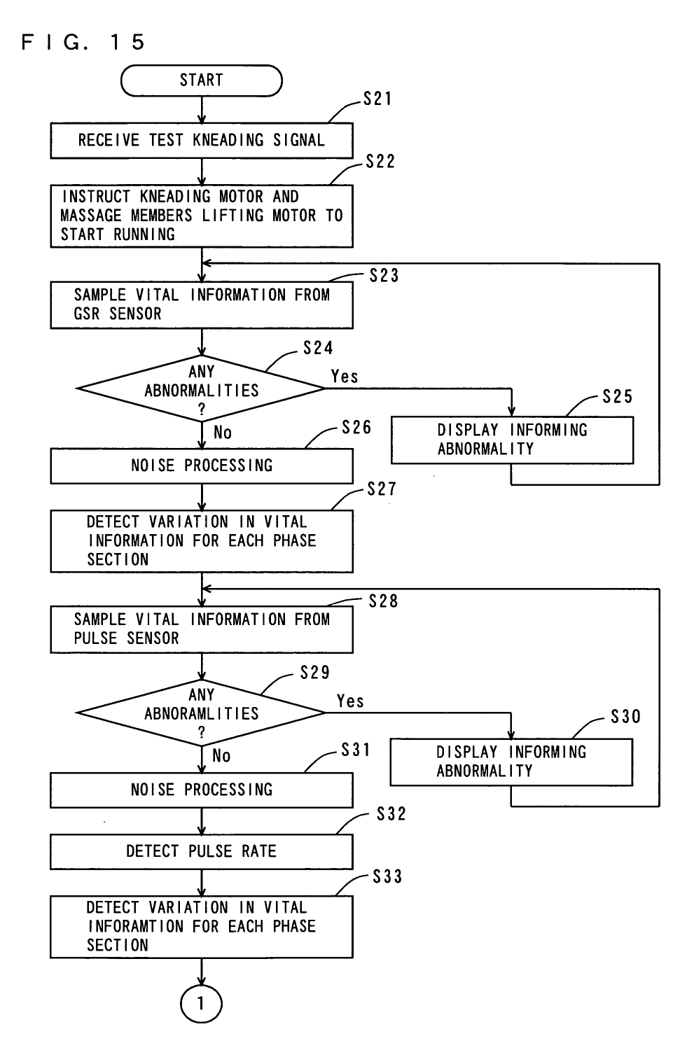

Now referring to FIG. 17, the control circuit 21 gives an instruction to the air conditioner 500 based on the selected item of the air conditioning control table (Step S47), gives an instruction to the illuminator 510 based on the selected item of the illumination adjustment control table (Step S48), and gives an instruction to the video/audio apparatus 520 based on the selected item of the video/audio adjustment control table (Step S49).

The control table 21 subsequently makes the liquid crystal display 16 of the remote controller 10 display the determination result for each of the phase sections (Step S50).

The user sees the display on the liquid crystal display 16, and presses the start/stop switch 14.

The control circuit 21 determines whether or not the start/stop switch 14 has been pressed (Step S51). Upon determining that the start/stop switch 14 has not been pressed, the control circuit 21 waits until it is pressed.

On the other hand, upon determining that the start/stop switch 14 has been pressed, the control circuit 21 instructs the massage members lifting motor 24 and the kneading motor 25 to start running based on the selected and adjusted item of the kneading pattern table (Step S52).

This causes the massage operation to start based on the adjusted kneading pattern table.

In the embodiment, the massage by the massager 100 is started after giving instructions to the air conditioner 500, illuminator 510, and video/audio apparatus 520 based on the respective operating tables; note, however, that the massage by the massager 100 may be started prior to giving instructions to the air conditioner 500, illuminator 510, and video/audio apparatus 520 based on the respective operating tables. Still alternatively, the air conditioner 500, illuminator 510, and video/audio apparatus 520 may be given instructions based on different operating tables for each of the phase section.

After this, the control circuit 21 determines whether or not a given period of time has elapsed (Step S53). The control circuit 21 continues massaging until the given period of time elapses. On the other hand, upon determining that the given period of time has elapsed, the control circuit 21 instructs the massage members lifting motor 24 and the kneading motor 25 to stop running (Step S54).

The operation of the control circuit 21 of the massager 100 according to the relaxation control program is thus completed.

As disclosed above, the relaxation system according to the embodiment, in which the massage operation is controlled based on the average value of the momentum variations ΔF of the human body, can help recovery from fatigue more effectively in a short period of time, according to the average value of the momentum variations ΔF. In addition, since the speed and time of the massage members 30 are controlled based on the average value of the momentum variations ΔF, massage can be performed more effectively in a short period of time, according to the average value of the momentum variations ΔF.

Moreover, the air conditioner 500, illuminator 510, and video/audio apparatus 520 are controlled by the control circuit 21 of the massager 100 based on the average value of the momentum variations ΔF of the human body; and therefore, stress is visually relieved by the video/audio apparatus 520 and illuminator 510, audibly relieved by the video/audio apparatus 520, and physically relieved by controlling the temperature, humidity, and air velocity with the air conditioner 500. As a result of this, not only the physical fatigue but also the mental fatigue can be recovered.

Further, selection for the kneading pattern table is done based on the vital information, followed by adjustment of the selected massage operation based on the average value of the variations ΔF of the human body; and therefore, the relaxation program according to the embodiment can help recovery from fatigue more effectively in a shorter period of time, considering the current body condition and fatigue parts or degree of fatigue of the human body.

Furthermore, detection of the vital information using the GSR sensor 11, pulse sensor 12, and skin temperature sensor 13 enables an accurate detection of the degree of stress that the human body currently feels.

In the embodiment, detection of the acceleration data is done by the calorie meter 50, and calculation of the value of a momentum variation ΔF is done by the massager 100 using the estimated equations; note, however, that the value of a momentum variation ΔF may be calculated by the calorie meter 50 instead for transmission to the massager 100.

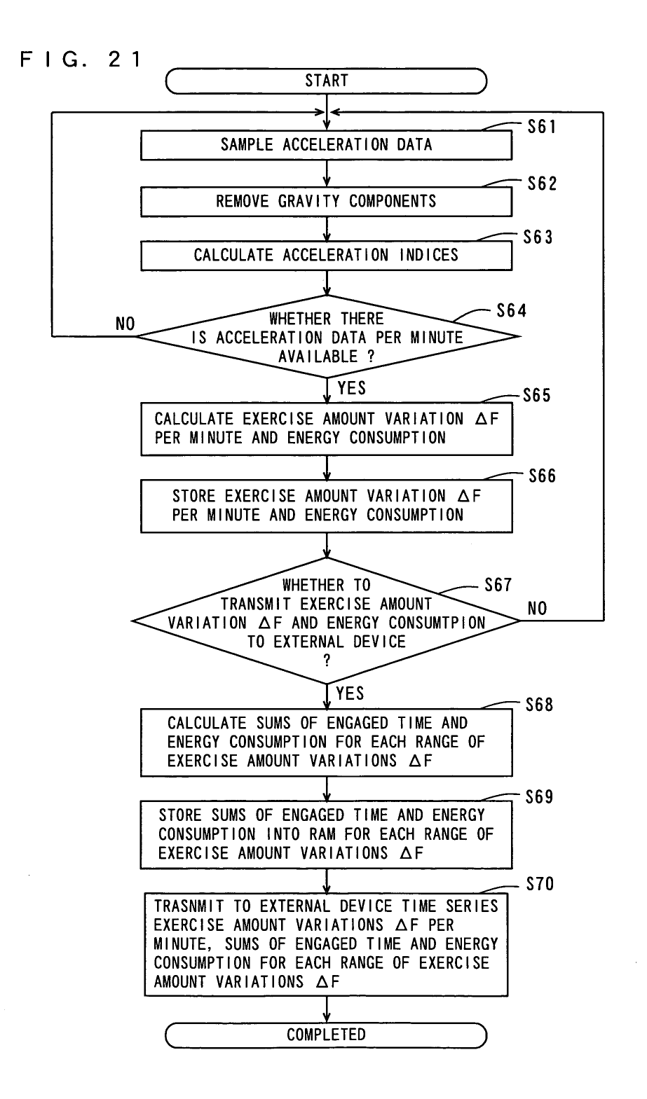

FIG. 21 is a flowchart showing another example of the operation of the CPU 56c according to the acceleration detection program during sampling of acceleration data. The user conducts physical activities or manual labor with the calorie meter 50 put on.

Initially, the CPU 56c of the calorie meter 50 samples acceleration data from the acceleration measurement device 51b of the calorie meter 50 (Step S61). In this case, the user is not wearing the portable indirect calorimetry system.

Then, the CPU 56c removes gravity components from the plurality of pieces of acceleration data (Step S62). The process of removing gravity components from the pieces of acceleration data is similar to that as described above. The CPU 56c subsequently calculates acceleration indices Acci (Step S63).

After this, the CPU 56c determines whether or not there is acceleration data per minute available (Step S64). For example, with the sampling cycle Fs of 25 Hz, it determines whether or not 1500 pieces of acceleration data have been sampled. Upon determining that there is no acceleration data per minute available, the CPU 56c returns to Step S61 to repeat the processes of Step S61 to Step S64.

On the other hand, upon determining that there is the acceleration data per minute available, the CPU 56c calculates a momentum variation ΔF and energy expenditure per minute based on the acceleration indices Acci measured for one minute (Step S65). Here, the momentum variation ΔF per minute can be obtained according to the equation below:
ΔF=ΣAcci/W
where Σ Acci represents the sum of the acceleration indices Acci obtained by one-minute long sampling, and W represents the weight of the user. The energy expenditure can be found based on the momentum variations ΔF, using the estimated equations previously obtained from above FIG. 13.

Then, the CPU 56c makes the RAM 56a store the calculated momentum variation ΔF and energy expenditure per minute (Step S66).

After this, the CPU 56c determines whether or not the user has ordered the transmission of the momentum variation ΔF and the energy expenditure (Step S67). When the user has not ordered the transmission, the CPU 56c returns to Step S61 to repeat the processes of Step 61 to Step 67 for each minute.

When, on the other hand, the user has ordered the transmission of the momentum variation ΔF and the energy expenditure, the CPU 56c calculates the sums of engaged time and energy expenditure for each range of momentum variations ΔF, using the momentum variations ΔF and energy expenditure per minute stored in the RAM 56a (Step S68).

Each of the ranges of the momentum variations ΔF is, for example, a range of 0≦ΔF≦2, a range of 2≦ΔF≦9, a range of 9≦ΔF≦15, a range of 15≦ΔF≦25, a range of 25≦ΔF≦30, and a range of 30≦ΔF. Note that the term engaged time refers to the time in which the user conducts physical activities with the calorie meter 50 put on.

The CPU 56c makes the RAM 56a store the sums of calculated engaged time and energy expenditure, for each range of the momentum variations ΔF (Step S69), and subsequently, transmits to an external device the time series momentum variations ΔF per minute, the sums of engaged time and the energy expenditure for each of the ranges of momentums variations ΔF (Step S70). The external device as used here in may for example be the massager 100, air conditioner 500, illuminator 510, video/audio apparatus 520, personal computer, or the like.

The external device is capable of determining the active part of body, working activity, and amount of activity, based on the time series momentum variations ΔF per minute, the sums of the engaged time and the energy expenditure for each of the ranges of momentum variations ΔF that have been received from the calorie meter 50.

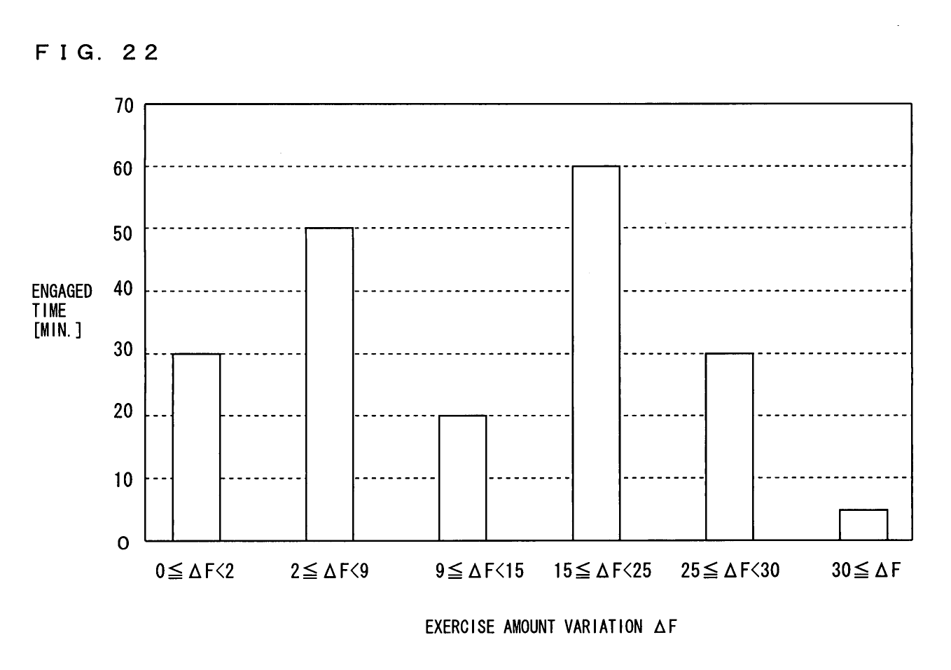

FIG. 22 is a graph showing an example of the sum of the engaged time for each of the ranges of the momentum variations ΔF calculated by the calorie meter 50.

In the example of FIG. 22, the engaged time is the longest in the range of 15≦ΔF<25, and the second longest in the range of 2≦ΔF≦9.

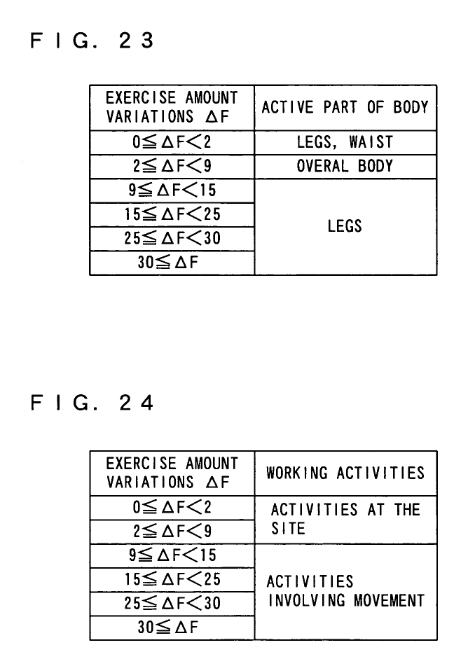

FIG. 23 shows an example of an active body part determination table representing the relationship between each of the ranges of momentum variations ΔF and the active part of body.

The active body part determination table has been created in advance during the formation of parameters for storage into a memory such as a RAM in the external device. In the example of FIG. 23, when the value of the momentum variation ΔF is not less than 0 and less than 2, the active part of body is determined as the waist; when the value is not less than 2 and less than 9, the active part of body is determined as the overall body; and when the value is not less than 9, the active part of body is determined as the legs.

FIG. 24 shows an example of a working activity determination table representing the relationship between each of the ranges of momentum variations ΔF and the working activity.

The working activity determination table has been created in advance during the formation of parameters for storage into a memory such as a RAM in the external device. In the example of FIG. 24, when the values of the momentum variations ΔF are not less than 0 and less than 9, the working activities are determined as the activities conducted at the site. When, on the other hand, the values are more than 9, the working activities are determined as the activities involving movement. In this case, the determination threshold for the working activities is 9.

The external device is capable of determining the active part of body using the active body part determination table of FIG. 23, based on the average value of the momentum variations ΔF per minute received from the calorie meter 50. For example, when the average value of the momentum variations ΔF per minute obtained from the calorie meter 50 is 10, the active part of body is determined as the legs.

In addition, the external device is capable of determining the working activity using the working activity determination table of FIG. 24, based on the average value of the momentum variations ΔF per minute received from the calorie meter 50. For example, when the average value of the momentum variations ΔF per minute obtained from the calorie meter 50 is 10, the working activity is determined as the activity involving movement.

It is also possible to determine the amount of activity conducted by each active part based on the sum of the engaged time for each of the ranges of the momentum variations ΔF obtained from the calorie meter 50. For example, when the engaged time is long in the range of 15≦ΔF≦25, the amount of activity for the legs is determined to be great.

It is also possible to determine the amount of activity conducted by each active part based on the sum of the energy expenditure for each of the ranges of the momentum variations ΔF obtained from the calorie meter 50. For example, when the engaged time is long in the range of 15≦ΔF≦25, the amount of activity for the legs is determined to be great.

Thus, the external device functions as the physical activity determiner which determines the active part of body, working activity, and amount of activity.

Determination results of the active part of body, working activity, and amount of activity are displayed on the display unit of the external device. As an alternative, the calorie meter 50 may determine the active part of body, working activity, and amount of activity based on the time series momentum variations ΔF per minute, the sums of the engaged time and the energy expenditure for each of the ranges of momentum variations ΔF. The calorie meter is preferably provided with a display unit for displaying the determination results of the active part of body, working activity, and amount of activity. In this case, the calorie meter 50 functions as the physical activity determiner.

The operations of the massager 100, the air conditioner 500, the illuminator 510, and the video/audio apparatus 520 can be controlled based on the determination results of the active part of body, working activity, and amount of activity.

For example, when the user keeps standing on his feet, the energy expenditure does not increase whereas the load on his feet and waist is considerable. In such a case, based on the determination results of the active parts of body, working activity, and amount of activity, an intensive massage can be provided for the feet and waist.

It is thus possible to help recovery from fatigue appropriately, effectively, by performing an intensive massage for the parts determined as the active parts.

It is also possible to improve the efficiency of the working contents by determining the active part of body, working activity, and amount of activity during manual labor.

As an example, when the engaged time in the range of 0≦ΔF≦2 is 30 minutes or longer, it is determined that the user has kept on his feet for 30 minutes or longer. In this case, the working person can be encouraged a break. This results in more efficient working contents.

In the embodiment, the calorie meter 50 corresponds to an acceleration measurement device, the massager 100 corresponds to a relaxation apparatus and a massage apparatus, the control circuit 21 or the CPU 56c corresponds to an estimator or a determiner, the control circuit 21 or the CPU 56c corresponds to a control unit, the massage members 30 correspond to pressing members, the massager 100, the air conditioner 500, the illuminator 510, or the video/audio apparatus 520 corresponds to a relaxation apparatus, the GSR sensor, pulse sensor 12 or the skin temperature sensor 13 corresponds to a detector.

In the above embodiment, the massager 100, air conditioner 500, illuminator 510, and video/audio apparatus 520 are used as the relaxation apparatus; note, however, that various kinds of relaxation apparatus may be used instead. For example, a Jacuzzi bath, a fragrance such as an aroma, or the like may be used as the relaxation apparatus, with the pressure of the Jacuzzi bath, the supply amount of the fragrance or the like being controlled based on the momentum variations ΔF.

Although the present invention has been described and illustrated in detail, it is clearly understood that the same is by way of illustration and example only and is not to be taken by way of limitation, the spirit and scope of the present invention being limited only by the terms of the appended claims.

1. A relaxation system comprising:
an acceleration measurement device that is attachable to a human body, and measures the acceleration of an attached part;
a relaxation apparatus that performs a relaxation operation;
an estimator that estimates activity information of the human body based on the acceleration measured by said acceleration measurement device; and
a controller that controls said relaxation apparatus based on a result of estimation by said estimator.

2. The relaxation system according to claim 1, wherein
said activity information includes an active part of the human body.

3. The relaxation system according to claim 1, wherein
said estimator calculates a momentum variation based on the acceleration measured by said acceleration measurement device, to estimate the active information of the human body based on the calculated momentum variation.

4. The relaxation system according to claim 1, wherein
said relaxation apparatus includes a massage apparatus with a pressing member movably provided in order to press parts of the human body, and
said controller controls at least either of the speed or time of said pressing member based on the result of estimation by said estimator.

5. The relaxation system according to claim 1, wherein
said relaxation apparatus relieves stress visually, audibly or physically.

6. The relaxation system according to claim 5, wherein
said relaxation apparatus includes one or more of an air conditioner, an audio apparatus, a video display, and an illuminator, and
said controller controls said one or more of air conditioner, audio apparatus, video display, and illuminator based on the result of estimation by said estimator.

7. The relaxation system according to claim 1, further comprising a detector that detects vital information of the human body, wherein
said controller sets the operation of said relaxation apparatus based on the vital information detected by said detector, to adjust said set operation based on the result of estimation by said estimator.

8. The relaxation system according to claim 1, wherein
said detector includes at least one of a galvanic skin response sensor, a pulse sensor, and a skin temperature sensor.

9. The relaxation system according to claim 1, further comprising an attachment tool for use in attaching said acceleration measurement device around the waist of the human body.

10. A relaxation method comprising the steps of:
measuring the acceleration of a human body;
performing a relaxation operation;
estimating active information of the human body based on said measured acceleration; and
controlling said relaxation operation based on said result of estimation.

11. A computer-executable relaxation program that makes said computer to execute the processes of:
obtaining acceleration from an acceleration measurement device that measures the acceleration of a human body;
performing a relaxation operation;
estimating activity information of the human body based on said obtained acceleration; and
controlling said relaxation operation based on said result of estimation.

12. A massage system comprising:
an acceleration measurement device that is attachable to a human body, and measures the acceleration of an attached part;
a massage apparatus that performs a massage operation;
an estimator that estimates activity information of the human body based on the acceleration measured by said acceleration measurement device; and
a controller that controls said massage apparatus based on a result of estimation by said estimator.

13. The massage system according to claim 12, wherein said massage apparatus includes a pressing member movably provided in order to press parts of the human body, and said controller controls at least either of the speed or time of said pressing member based on the result of estimation by said estimator.

14. The massage system according to claim 12, further comprising a relaxation apparatus that relieves stress visually, audibly, and physically, wherein
said controller controls said relaxation apparatus based on the result of estimation by said estimator.

15. The massage system according to claim 14, wherein said relaxation apparatus includes one or more of an air conditioner, an audio apparatus, a video display, and an illuminator, and
said controller controls said one or more of air conditioner, audio apparatus, video display, and illuminator based on the result of estimation by said estimator.

16. The massage system according to claim 12, further comprising a detector that detects vital information of the human body, wherein
said controller sets the operation of said massage apparatus based on the vital information detected by said detector, to adjust said set operation based on the result of estimation by said estimator.

17. The massage system according to claim 16, wherein said detector includes at least one of a galvanic skin response sensor, a pulse sensor, and a skin temperature sensor.

18. A massage method comprising the steps of:
measuring the acceleration of a human body;
performing a massage operation;
estimating active information of the human body based on said measured acceleration; and
controlling said massage operation based on said result of estimation.

19. A computer-executable massage program that makes said computer to execute the processes of:
obtaining acceleration from an acceleration measurement device that measures the acceleration of a human body;
performing a massage operation;
estimating activity information of the human body based on said obtained acceleration; and
controlling said massage operation based on said result of estimation.

20. A physical activity determiner comprising:
an acceleration measurement device that is attachable to a human body, and measures the acceleration of an attached part; and
a determiner that determines activity information of the human body based on the acceleration measured by said acceleration measurement device.

21. The physical activity determiner according to claim 20, wherein
said determiner calculates a momentum variation based on the acceleration measured by said acceleration measurement device, to determine the active information of the human body based on the calculated momentum variation.

22. The physical activity determiner according to claim 21, wherein
said determiner stores in advance the relationship between momentum variations and active information of the human body, to determine the activity information of the human body based on said calculated momentum variation with reference to said stored relationship.

23. A physical activity determination method comprising the steps of:
measuring the acceleration of a human body, and
determining the active information of the human body based on said measured acceleration.

24. A computer-executable physical activity determination program that makes said computer execute the processes of:
obtaining acceleration from an acceleration measurement device that measures the acceleration of a human body; and
determining active information of the human body based on said obtained acceleration.
